(19) United States
(12) Patent Application Publication
(10) Pub. No.: US 2022/0047630 A1
(43) Pub. Date: Feb. 17, 2022

(54) BARN DUST EXTRACT FOR THE PREVENTION AND TREATMENT OF DISEASES

(71) Applicants: Helmholtz Zentrum München - Deutsches Forschungszentrum Für Gesundheit Und Umwelt (GmbH), Neuherberg (DE); The Arizona Board of Regents on Behalf of the University of Arizona, Tucson, AZ (US)

(72) Inventors: Erika Von Mutius, München (DE); Franz Bracher, München (DE); Christoph Müller, München (DE); Donata Vercelli, Tucson, AZ (US)

(21) Appl. No.: 17/413,468

(22) PCT Filed: Dec. 13, 2019
(86) PCT No.: PCT/EP2019/085016

§ 371 Date: Jun. 11, 2021

(30) Foreign Application Priority Data
Dec. 14, 2018 (LU) 101064

(51) Int. Cl. A61K 35/02; A61P 37/08

(52) U.S. Cl. CPC A61K 35/02; A61P 37/08

(57) ABSTRACT

The present invention relates to a method of preparing a barn dust extract, said barn dust extract, and a composition comprising said barn dust extract. The barn dust extract is useful in the prevention or treatment of a disease.

### FIELD OF THE INVENTION

The present invention relates to a method of preparing a barn dust extract, said barn dust extract, and a composition comprising said barn dust extract. The barn dust extract is useful in the prevention or treatment of a disease.

### BACKGROUND

Allergy has developed into a major health concern in Europe with over 80 million people affected by some form of allergic disease and around 30 million people suffering from asthma. About 50% of all children (in some areas even 70%) have a positive allergy test to ‘normal’ exposures such as house dust, animals, pollen and food. The human immune system has become less able to tolerate these natural exposures and instead reacts with immunoglobulin E (IgE) antibodies, therefore developing allergic sensitization. These overshooting immune responses result in allergic airway disease such as hay fever and allergic asthma. The prevalence of hay fever is 20% among school children and 30% among teenagers. About 10% of children suffer from asthma. Most allergic patients develop a chronic course spreading into adolescence and adult age which results in significant health threats and severe limitations of activities and quality of life.

A way to prevent the new onset of asthma, hay fever and allergic sensitization is yet to be found, however there is compelling and consistent evidence across more than 30 studies worldwide that children growing up in a farming environment rich in microbial exposures are better protected from developing asthma, allergic rhinitis and atopic sensitization.

In the ALEX study the differences between farm and nonfarm children were: 1.4% versus 11.8% asthma, 3.2% versus 16% hay fever, 12.4% versus 32.9% allergic sensitization. This protection is sustained into adult life (von Mutius E and Vercelli D. Farm living: Effects on childhood asthma and allergy. Nat. Rev. Immunol. 10: 861-868 (2010) PMID: 21060319).

The farm effect on asthma can be explained by the child's early life contact to farm animals, mostly cattle (Illi S, Depner M, Genuneit J, et al. Protection from childhood asthma and allergy in Alpine farm environments—the GABRIEL Advanced Studies. J Allergy Clin Immunol 2012;129:1470-7 e6; Loss G J, Depner M, Hose A J, et al. The Early Development of Wheeze. Environmental Determinants and Genetic Susceptibility at 17q21. Am J Respir Crit Care Med 2016;193:889-97). These findings suggest that exposures encountered in animal sheds, in particular cattle stables, play a major role. Importantly, the epidemiological findings have been translated into experimental studies in mice thereby adding biological validity to these observations. In two independent studies, dust from a Bavarian cow shed (Schuijs MJ, Willart MA, Vergote K, et al. Farm dust and endotoxin protect against allergy through A20 induction in lung epithelial cells. Science 2015;349:1106-10) and aqueous extract of cow shed dust collected in the ALEX study (Peters M, Kauth M, Schemer O, et al. Arabinogalactan isolated from cowshed dust extract protects mice from allergic airway inflammation and sensitization. J Allergy Clin Immunol 2010;126:648-56 e1-4) were administered in aerosolized form (Peters M, Kauth M, Schemer O, et al. Arabinogalactan isolated from cowshed dust extract protects mice from allergic airway inflammation and sensitization. J Allergy Clin Immunol 2010;126:648-56 e1-4) or intranasally (Schuijs MJ, Willart MA, Vergote K, et al. Farm dust and endotoxin protect against allergy through A20 induction in lung epithelial cells. Science 2015;349:1106-10) in ovalbumin (OVA)-induced allergic asthma model (Peters M, Kauth M, Schemer 0, et al. Arabinogalactan isolated from cowshed dust extract protects mice from allergic airway inflammation and sensitization. J Allergy Clin Immunol 2010;126:648-56 e1-4) or house dust mite (Schuijs M J, Willart M A, Vergote K, et al. Farm dust and endotoxin protect against allergy through A20 induction in lung epithelial cells. Science 2015;349:1106-10) models of allergic asthma. In all studies this prophylactic treatment resulted in dramatically reduced airway hyperresponsiveness, suppressed eosinophilia and reduced levels of IgE, all hallmarks of allergic asthma.

Allergy therapies must often be administered over years and decades, and the application of newly developed biologicals results in exploding costs, therefore there is a need of developing preventive cures as they represent the only true long-term solution. Therefore, the technical problem underlying the present invention is the provision of further therapies for allergy development, in particular preventive therapies.

### SUMMARY OF THE INVENTION

The present invention relates to a method of preparing a barn dust extract, comprising: (d) providing a mixture comprising a barn dust and a liquid; and (e) isolating a fraction of the mixture, wherein the barn dust extract comprised in the fraction consists essentially of molecules having a molecular weight of at least about 5 kDa, preferably at least of about 10 kDa.

The present invention also relates to a barn dust extract obtainable by a method described herein.

The present invention also relates to a barn dust extract consisting to at least about 90% by dry matter of molecules having a molecular weight of at least about 5 kDa, preferably at least about 10 kDa, wherein a size exclusion chromatogram of the barn dust extract has in the fraction having a molecular weight of at least about 10 kDa two characteristic peaks A and B, wherein (a) peak A has its maximum in the range of 35 to 75 kDa (corresponding to a retention time of about 17 min, preferably as measured in an size exclusion chromatography assay as described herein); and (b) peak B has its maximum in the range of 300 to 700 kDa (corresponding to a retention time of about 11 min, preferably as measured in an size exclusion chromatography assay as described herein).

The present invention also relates to a composition comprising the barn dust extract of the invention, in particular a pharmaceutical composition.

The present invention also relates to a barn dust extract of the invention or a composition of the invention for use in the prevention or treatment of a disease.

The present invention also relates to a use of the barn dust extract of the invention for the manufacture of a composition for the prevention or treatment of a disease.

The present invention also relates to a method of preventing or treating a disease comprising administering a therapeutically effective amount of a barn dust extract of the invention to a subject.

### BRIEF DESCRIPTION OF THE DRAWINGS

FIG. 1 shows a HPLC chromatogram of the stable dust total extract that has been not lyophilized and not autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter: 4.6 mm, particle size 2.7 μm).

Column oven temperature: 30° C.

Mobile phase: 0.15 M NaCl with bidest. H₂O.

Flow rate 0.3 mL/min.

Injection volume: 10 μL.

FIG. 2 shows a HPLC chromatogram of the stable dust total extract that has been lyophilized and not autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter: 4.6 mm, particle size 2.7 μm).

Column oven temperature: 30° C.

Mobile phase: 0.15 M NaCl with bidest. H₂O.

Flow rate 0.3 mL/min.

Injection volume: 10 μL (12.3 mg/mL lyophilized dust in water).

FIG. 3 shows a chromatogram of the stable dust Total extract that has been lyophilized and autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter: 4.6 mm, particle size 2.7 μm).

Column oven temperature: 30° C.

Mobile phase: 0.15 M NaCl with bidest. H₂O.

Flow rate 0.3 mL/min.

Injection volume: 10 μL (9.9 mg/mL lyophilized dust in water).

FIG. 4 provides an overview allowing comparison of the chromatograms depicted in FIGS. 1, 2 and 3.

FIG. 5 shows a chromatogram of the >10 kDa fraction of a stable dust extract that has not been lyophilized and not autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter: 4.6 mm, particle size 2.7 μm).

Column oven temperature: 30° C.

Mobile phase: 0.15 M NaCl with bidest. H₂O.

Flow rate 0.3 mL/min.

Injection volume: 10 μL.

FIG. 6 shows a chromatogram of the >10 kDa fraction of a stable dust extract that has been lyophilized and not autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter: 4.6 mm, particle size 2.7 μm).

Column oven temperature: 30° C.

Mobile phase: 0.15 M NaCl with bidest. H₂O.

Flow rate 0.3 mL/min.

Injection volume: 10 μL (13.0 mg/mL lyophilized dust in water).

FIG. 7 shows a chromatogram of the >10 kDa fraction of a stable dust extract that has been lyophilized and autoclaved. Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter: 4.6 mm, particle size 2.7 μm).

Column oven temperature: 30° C.

Mobile phase: 0.15 M NaCl with bidest. H₂O.

Flow rate 0.3 mL/min.

Injection volume: 10 μL (8.4 mg/mL lyophilized dust in water).

FIG. 8 provides an overview allowing comparison of the chromatograms depicted in FIGS. 5, 6 and 7.

FIG. 9 illustrates the change in liquid extract (not autoclaved) measured after 8 days of storage under different conditions, namely storage at room temperature, refrigerated (storage at 4° C.) and freezed (storage at −20° C.)

FIG. 10 illustrates the change in peak height for the peaks of respectively 27 kDA, 5 kDA, 1 kDa, and 0.6 kDa, measured after 8 days of storage under different conditions, namely storage at room temperature, storage at 4° C. and storage at −20° C.

FIG. 11 illustrates the difference between non-autoclaved and autoclaved liquid extracts obtained from barn dust from Hechfellner Hof.

FIG. 12 illustrates the change in liquid extract (autoclaved) measured after 5 days of storage under different conditions, namely storage at room temperature, storage at 4° C. and storage at −20° C.

FIG. 13 illustrates the change in liquid extract (autoclaved) measured after 12 days of storage under different conditions, namely storage at room temperature, storage at 4° C. and storage at −20° C.

FIG. 14 illustrates the change in peak height for the peaks of respectively 309 kDa, 6 kDa, 2 kDa, and 0.8 kDa, measured after 8 days of storage under different conditions, namely storage at room temperature, storage at 4° and storage at −20° .

FIG. 15 illustrates the experimental setup used to measure eosinophil recruitment to the lung in mice after intra-nasal administration of fractionated or unfractionated stable dust extracts. Dust extracts (0.8 mg dry weight in 50 μl/treatment) are instilled intra-nasally every 2-3 days for a total of 8 times beginning at day 0 into 7-week old Balb/c mice that are sensitized intra-peritoneally with 50 μg of OVA-Alum (6 mg) at day 0 and 7, and challenged intra-nasally with 100 μg OVA at day 14, 15, and 16. Broncho-alveolar lavage (BAL) is performed at day 17 by delivering cold 1% BSA in PBS (2 mL) into the airway via a tracheal cannula and gently aspirating the fluid. Cells are counted using a Countess II FL automated cell counter (Thermo Fisher Scientific) and differentials are determined by an operator blinded to mouse ID/grouping after staining with Hema 3 (Fischer) (at least 400 cells/slide). Statistical significance for BAL cellularity measurements is determined by an unpaired two tailed Student's t-test. P-values <0.05 are considered significant. Each treatment group includes a minimum of 5 mice

Left panel of FIG. 16 illustrates eosinophils count after treating mice with a saline solution (negative control), an allergen (OVA, positive control), unfractionated, autoclaved stable dust extract (BAV Autoclaved), and unfractionated, non-autoclaved stable dust extract (BAV Non-Autoclaved).

In the right panel are reported eosinophils counts after treating mice with a saline solution (negative control), an allergen (OVA, positive control), unfractionated, autoclaved barn dust extract (Bay 0/0), fractions between 10 kDa and 100 kDa of autoclaved stable dust extract (Bay 100/10), and fractions bigger than 10 kDa of autoclaved stable dust extract (Bav >10).

FIG. 17 illustrates the experimental setup used to measure proportions of γδ T cells in the lungs of mice following intra-nasal administration of a saline solution (negative control), an allergen (OVA, positive control), unfractionated, autoclaved barn dust extract, fractions between 10 kDa and 100 kDa of autoclaved stable dust extract, and fractions bigger than 10 kDa of autoclaved stable dust extract.

Mice are euthanized by lethal dose of anesthetic. Lungs are perfused and removed. To prepare single-cell suspension the lungs are minced with scissors and digested with 0.26 Wunsch U/ml of Liberase TM (Roche) and 4 U/ml of DNAse I (Sigma) at 37° C. for 1 h with shaking (60-70 rpm). The resulting suspension is passed through 20-22 G needle and through the 70 μtm cell strainer. After washing with complete medium, lung cells are resuspended in PBS/0.1% NaN3/1.0% BSA at 5×10⁶ cells/ml. Cells are treated with Fc-Block for 10 min on ice and stained with fluorescently labeled antibodies to cell surface antigens for 30 min on ice in the dark. Cells are then washed and resuspended in PBS/0.1% NaN3/1.0% BSA for flow cytometry.

The following antibodies to cell surface antigens are used: PE or BV510 conjugated hamster anti-mouse γδ T-Cell Receptor (GL3), FITC-hamster anti-mouse CD3c (145-2C11), PE-Cy7 or PE-Cy5-rat anti-mouse CD4 (RM4-5), APC-R700-rat anti-mouse CD8α (53-6.7). All from BD Pharmingen. Flow cytometry is performed on a FACSCalibur or LSRII (BD Sciences) flow cytometers. Data is collected on 10000-20000 events. Data analysis is performed using CellQuest (BD) or FlowJo (FlowJo) software. Proportions of γδ T cells are reported as % of CD3+ T cells gating on lung lymphocytes.

FIG. 18 illustrates the results of flow cytometry analysis to determine the proportion of CD3⁺γδ⁺ cells found in the lungs after treating mice with a saline solution (negative control). CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse.

FIG. 19 illustrates the results of flow cytometry analysis to determine the proportion of CD3⁺γδ⁺ cells found in the lungs after treating mice with an allergen (OVA). CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse

FIG. 20 illustrates the results of flow cytometry analysis to determine the proportion of CD3⁺γδ⁺cells found in the lungs after treating mice with allergen (OVA)+unfractionated, autoclaved barn dust extract. CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse

FIG. 21 illustrates the results of flow cytometry analysis to determine the proportion of CD3⁺γδ⁺ cells found in the lungs after treating mice with allergen (OVA)+fractions of autoclaved stable dust extract between 10 kDa and 100 kDa. CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse.

FIG. 22 illustrates the results of flow cytometry analysis to determine the proportion of C3⁺γδ⁺ cells found in the lungs after treating mice with allergen (OVA)+fractions of autoclaved stable dust extract >10 kDa. CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse.

### DETAILED DESCRIPTION

The present invention is based on the surprising finding that certain processing step(s) may improve the quality and/or anti-allergenic potential of a barn dust (extract). The inventors of the present application have surprisingly found that certain fractions of barn dust extracts obtained after fractionation according to molecular weight of barn dust components exhibit an improved anti-allergenic potential as compared to the whole extract.

According to the surprising finding the present invention relates to a method of preparing a barn dust extract, comprising: (d) providing a mixture comprising a barn dust and a liquid; (e) isolating a fraction of the mixture, wherein the barn dust extract comprised in the fraction consists essentially of molecules having a molecular weight of at least about 5 kDa, preferably at least about 10 kDa.

The term “barn dust” as used herein, relates to dust that can be, is, or has been collected from a barn. Without wishing to be bound by theory it is believed that barn dust comprises immunostimulatory substances derived from microorganisms, animals, plants, fungi, viruses and/or protozoa that are protective against allergies, asthma and/or other diseases disclosed herein. In preferred embodiments, the barn dust is from a farm. The origin of the barn dust is not limited to certain types of barns and can be any type of barn, including barns for any type of livestock such as cows, pigs, chicken, sheep, horse, or others. Dust from cow barns are however preferred. Also, the geographic location of the barn is believed to be not essential for the invention. Exemplary non-limiting geographic locations for a barn are Europe, including the member states of the European Union, such as Germany, France, Austria, Switzerland, Czech Republic, Poland, the Netherlands, Belgium, Luxemburg, Spain, Portugal, Italy, etc., the barn may for example be located in Bavaria, Germany. As a non-limiting example, barn dust may be obtained from Hechfellner Hof, Mettenheim, Bavaria, Germany. Barn dust can be collected by any suitable method known to the person skilled in the art optionally by applying any type of collection system that is suitable for collecting barn dust. Barn dust can for example be collected by sweeping, vacuuming, or swiping. Barn dust can also be collected by filtration of barn air, for example by using the membrane filter or a granular material that is capable of adsorbing barn dust. Barn dust can also be collected using an impinge or an impactor, such as a cascade impactor.

After collection, the dust may be homogenized. A suitable type of technique is any technique which leads to a uniform homogenization of the dust, in particular one which removes agglomerations and lumps of the dust. Suitable for these purposes are methods such as rubbing, smashing or crushing, stirring or introducing into a blender, without being limited thereto. Digesting the dust may also be part of the process according to the invention. Digesting of the constituents present in the dust, such as cells, microorganisms, in particular their spores and the like can be effected, for example by grinding, squashing and similar methods. A sieving step for removing (large) particles, such as particles having a size of about 100 μm, about 90 μm, about 80 μm, about 70 μm, about 60 μm, about 50 μm, about 40 μm, about 30 μm, about 20 μm, or about 10 μm may be applied after homogenization.

The term “barn dust extract” as used herein preferably refers to a composition that is obtainable by the methods disclosed herein and may refer to both, a solution or suspension, or a dry composition.

For the methods of the present invention the barn dust is optionally in a mixture with a liquid. The liquid may be water, an aqueous solution, or a water miscible solvent, or a combination thereof. It can be pure water, but it can also be a saline solution. A saline solution may contain sodium salts or similar monovalent or divalent salts, such as sodium sulfate, potassium chloride, magnesium chloride, or calcium chloride. A preferred saline solution is a solution of sodium chloride, preferably a physiologic aqueous solution of sodium chloride, such as normal saline. The term “normal saline” as used herein preferably refers to a mixture of sodium chloride and water at a concentration of about 9 g/L. The aqueous solution may also comprise a buffer, such as phosphate buffered saline. The water-miscible solvent may for example be an alcohol such as ethanol. The water-miscible solvent may also be a mixture of water-miscible solvents. The liquid may also be a mixture of water and/or an aqueous solution and one or more water-miscible solvent(s).

The term “fraction” as used herein preferably refers to fractions that can be obtained by fractioning a mixture according to the molecular weight and/or size of the molecules comprising the mixture.

The term “isolating” or “isolation” as used herein preferably refers to enriching a portion that is to be isolated as compared to a portion that is not to be isolated by mass, or depleting a portion that is not to be isolated as compared to a portion that is to be isolated by mass. Accordingly, step (e) of the methods of the present invention may comprise enriching a fraction wherein the barn dust extract comprised therein consists essentially of molecules having a molecular weight of at least about 5 kDa, preferably at least about 10 kDa by factor of at least about 5, preferably at least about 10, preferably at least about 20, preferably at least about 100. Step (e) of the methods of the present invention may also comprises depleting a fraction wherein the barn dust extract comprised therein consists essentially of molecules having a molecular weight of at most about 5 kDa, preferably at most about 10 kDa by factor of at least about 5, preferably at least about 10, preferably at least about 20, preferably at least about 100.

The term “consists essentially of” means that further components, such as impurities, can be present but that the further components do not markedly affect the essential characteristics of the components that the mixture, fraction, compound etc. essentially consists of. Such further component(s) may amount to up to about 10%, up to about 9%, up to about 8%, up to about 7%, up to about 6%, up to about 5%, up to about 4%, up to about 3%, up to about 2%, up to about 1%, up to about 0.5%, up to about 0.2%, or up to about 0.1% by weight of component(s) that the mixture, fraction, compound etc. essentially consists of.

The inventors of the present application have also surprisingly found that isolating a fraction, in which the barn dust comprised therein consists essentially of molecules having a molecular weight of at least about 5 kDa, preferably at least about 10 kDa, will yield a highly standardized product that is essentially independent from the origin of the starting material.

The method of the present invention may further comprise the step of subjecting a mixture comprising a barn dust and liquid to heat treatment. The heat treatment may be at a temperature of at least about 110° C. and/or a pressure of at least about 1.5 bar for at least about 3, 4, 5, 6, 7, 8, 9, or 10 minutes. The heat treatment may comprise subjecting the mixture to a moist heat, such as a saturated steam. The heat treatment may comprise autoclaving the mixture. Autoclaving may be performed using steam heated to about 121-134° C., for at least about 3 minutes, such as at least about 5 minutes, at least about 8 minutes, at least about 10 minutes, at least about 15 minutes or at least about 20 minutes. Such a heat treatment may be performed for up to about 2 hours, up to about 1.5 hours, up to about 1 hour, up to about 45 min or up to about 30 min. A preferred heat treatment may be subjecting the mixture to moist heat at 121° C. (2.1 bar) for at least about 15 minutes (such as about 20 minutes), at 134° C. (3 bar) for at least about 3 minutes (such as about 5 minutes), or at 134° C. (3 bar) for at least about 18 minutes. The latter conditions may further inactivate prions. Heat treatment can also be performed using dry heat, for example at about 160° C. to about 200° C. for up to about two hours. Optimal duration for dry heat treatment may vary depending of the temperature. Typical conditions for dry heat treatment are for example about 2 h at about 160 ° C., or about 1 h at about 170° C., or about 6 to 12 minutes at 190° C.

The inventors of the present application have surprisingly found that heat treatment alters the molecular weight distribution of the molecules comprised in the barn dust extract. However, it has surprisingly been found that this altered molecular weight distribution does not essentially affect the therapeutic potency of the barn dust extract. However, heat treatment is believed to result in a sterile product that meets safety requirements for pharmaceuticals and also improves stability of the product. Alternatively or additionally, the barn dust extract may undergo sterile filtration. A sterile filtration step may be conducted before step (e). A sterile filtration step may also be conducted before step (g).

The methods of the present invention may further comprise step (g), drying the fraction obtained in step (e). “Drying” as used herein preferably refers to complete or partial removal of solvents, in particular water. The drying step can be performed by any means or methods known to the person skilled in the art. Non-limiting examples include lyophilization, spray drying, sun drying, or air drying. Preferred methods are lyophilization or spray drying. A dry barn dust extract is believed to be more stable than a liquid extract and is thus particularly suited for long-term storage.

The methods of the present invention may comprise the step of (a) collecting barn dust. This collecting step can be performed by any method described here in. After collection of the barn dust, the barn dust may be mixed with a liquid described herein.

After mixing the dust with the liquid, this suspension may be either left to stand or stirred, so that the soluble substances, or the substances which can be removed from the dusts by the liquid, may enter into the liquid phase.

Thereafter, insoluble substances may be subsequently removed from the mixture. Removal can for example be conducted by filtration or a sedimentation step. The removal step is not limiting for the process according to the invention; any removal method which is known to the skilled worker may be employed. Also, leaving the suspension to stand so that sedimentation of the solid constituents can be accomplished by the earth's gravity may be considered as being for the purposes of the invention. A preferred way of removing the solid constituents is centrifuging, whereafter the supernatant is removed from the sediment.

Subsequently, the methods of the present invention may comprise step (c) depleting particles having a size of at least about 0.2 μm, which may comprise one or more filtration steps. Particles having a size of at least about 0.2 μm may be depleted by factor of at least about 10, preferably at least about 100, preferably at least about 1000.

The inventors of the present application have further surprisingly found that barn dust extract fractions that consists essentially of molecules having molecular weight of at least about 5 kDa, preferably at least about 10 kDa with no defined upper limit for the molecular weight have a higher therapeutic potency than fractions that consist essentially of molecules having molecular weight about 5 kDa to about 100 kDa or about 10 kDa to about 100 kDa. The maximum molecular weight or size of the molecules comprised in the barn dust extract may thus only be limited by the solubility of the molecules in water or the cut-off value of a filtration step in the preparation of the barn dust extract. Such a cut-off value may e.g. about 0.2 μm. Accordingly, the methods of the present invention preferably do not comprise the step of depleting molecules having a molecular weight of at least about 100 kDa from the barn dust extract. Accordingly the barn dust extract that consists essentially of molecules having a molecular weight of at least about 5 kDa, preferably at least about 10 kDa, preferably also comprises molecules having a molecular weight of at least about 100 kDa.

The methods of preparing a barn dust extract of the present invention may therefore comprise steps of:

(a) (optionally) collecting barn dust;
(b) (optionally) mixing barn dust with a liquid;
(c) (optionally) depleting particles having a size of at least about 0.2 μm;
(d) providing a mixture comprising a barn dust and a liquid;
(e) isolating a fraction of the mixture, wherein the barn dust extract comprised in the fraction consists essentially of molecules having a molecular weight of at least about 5 kDa, preferably at least about 10 kDa; and
(f) (optionally) subjecting the mixture to heat treatment;
(g) (optionally) drying the fraction obtained in step (f).

The present invention further relates to a barn dust extract. The barn dust extract is preferably obtainable by a method of the invention.

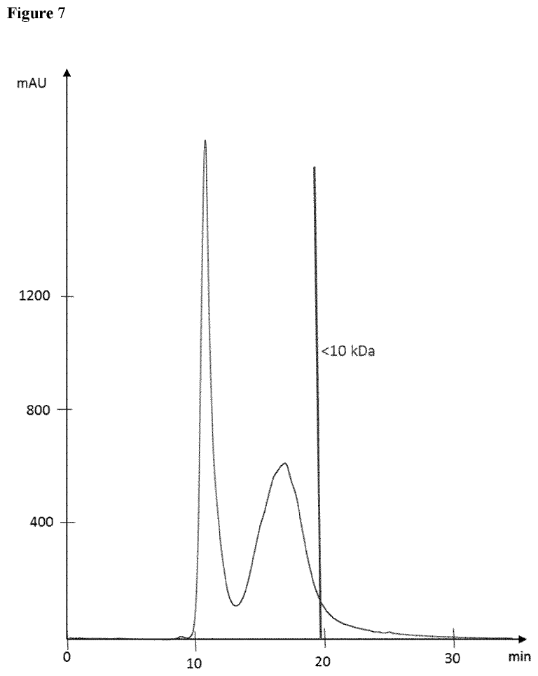

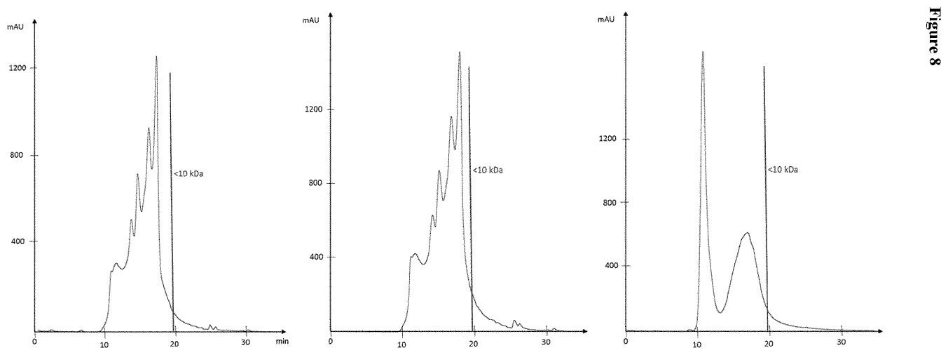

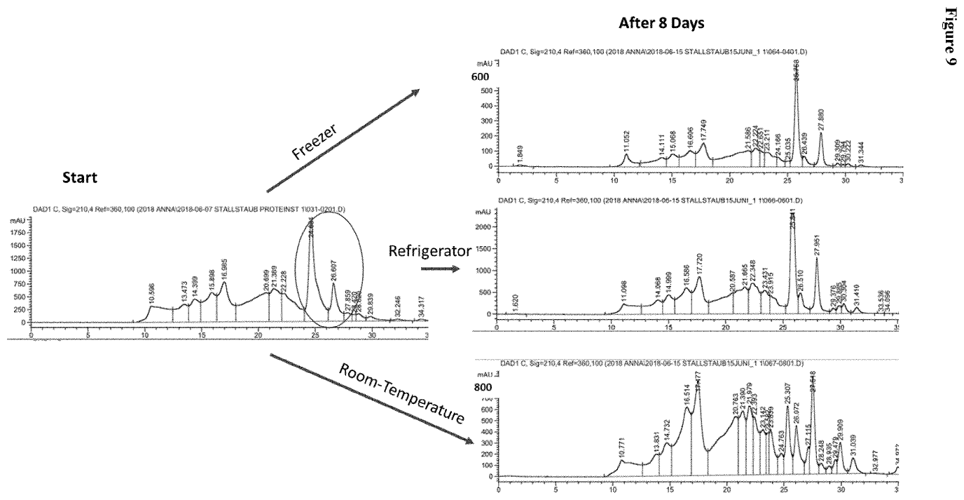

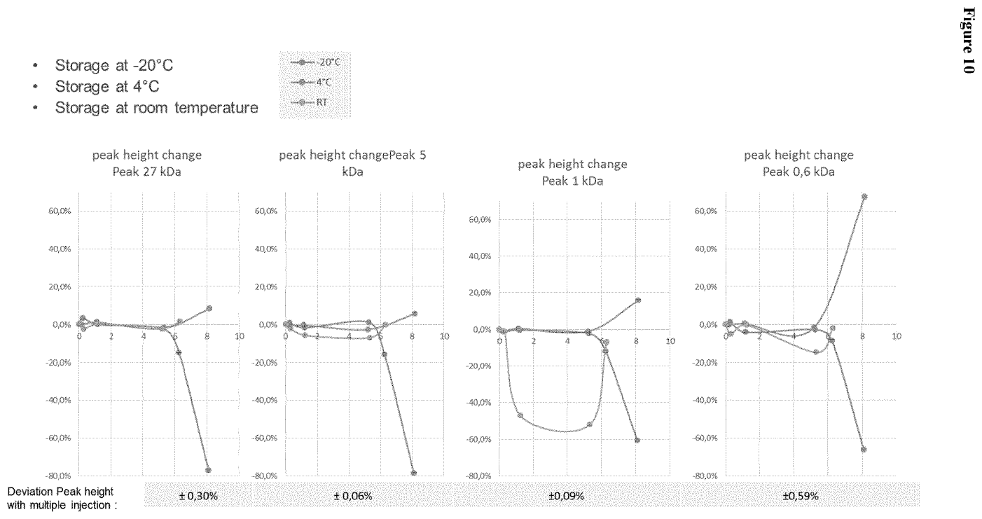

A barn dust extract of the invention may consist to at least about 90% by dry matter of molecules having a molecular weight of at least about 5 kDa, preferably at least about 10 kDa. Such a barn dust extract has a distinct molecular weight distribution of the molecules comprised in the extract. A size exclusion chromatogram of the barn dust extract has in the fraction having a molecular weight of at least about 10 kDa two characteristic peaks A and B, wherein peak A has its maximum in the range of 35 to 75 kDa (corresponding to a retention time of about 17 min, preferably as measured in an size exclusion chromatography assay as described herein); and (b) peak B has its maximum in the range of 300 to 700 kDa (corresponding to a retention time of about 11 min, preferably as measured in an size exclusion chromatography assay as described herein), as shown e.g. in FIG. 7.

Size exclusion chromatography is preferably carried out using an Agilent Advance Bio SEC 130 Å (2.7 μm, 4.6×300 mm) and/or an Agilent Advance Bio SEC 300 Å (2.7 μm, 4.6×300 mm) column and a 0.15 M NaCl solution as mobile phase, and wherein compounds are detected by an optical detector at a wavelength of 210 nm. Further conditions that are believed to be non-essential may be an injection volume (sample) of 10 μL, a column temperature of 30° C., and/or a flow rate of 0.3 mL/min.

The barn dust extract of the invention may have undergone heat treatment as defined in the methods of the invention.

The barn dust extract of the invention may consist essentially of molecules having a molecular weight of at least about 5 kDa, preferably at least about 10 kDa. Accordingly, the barn dust extract may consist consists to at least about 75%, preferably 80%, preferably 85%, preferably 90%, preferably 95%, preferably 98%, preferably 99%, preferably 99.5%, preferably 99.9% by dry matter of molecules having a molecular weight of at least about 5 kDa. The barn dust extract preferably consists to at least about 75%, preferably 80%, preferably 85%, preferably 90%, preferably 95%, preferably 98%, preferably 99%, preferably 99.5%, preferably 99.9% by dry matter of molecules having a molecular weight of at least about 10 kDa.

The barn dust extract disclosed herein preferably comprises one or more bacterial antigens. It can further comprise fungal, viral or protozoal antigens. A barn dust or barn dust extract disclosed herein may comprises a fragment of a bacterium selected from the group consisting of Staphylococcus sciuri, Jeotgalicoccus spp., preferably J. halotolerans, J. pinnipedialis, J. psychrophilus, Salinicoccus alkahphilus, Salinicoccus halodurans, Salinicoccus kunmingensis, Salinicoccus roseus, Macrococcusbrunensis, Duganella spp., preferably D. violaceinigra, D. zoogloeoides, Moraxella boevrei, Moraxella canis, Moraxella caprae, Moraxella cuniculi, Moraxella lincolnii, Corynebacterium variabile, Corynebacterium freiburgense, and Zooglea spp., preferably Z. caeni, Z. ramigera, Z. resimphila, or a mixture thereof. Alternatively or additionally, a barn dust or barn dust extract disclosed herein may comprise a fragment of a bacterium selected from the group consisting of Lactobacillus spp., preferably Lactobacillus curvatus, Lactobacillus sakei, and Lactobacillus iners, Delftia spp., preferably D. tsuruhatensis, Brevibacterium spp., preferably B. iodinum, B. linens, Rhizobium spp., preferably R. gallicum, Psychromonas spp., Alteromonas spp., Lactococcus lactis, and Acinetobacter spp., preferably Acinetobacter lwoffii or mixtures thereof.

The present invention also relates to a composition comprising a barn dust extract of the present invention.

Such a composition may be a pharmaceutical composition comprises the barn dust extract as active ingredient and optionally, one or more pharmaceutically excipient(s). Accordingly, the use of a barn dust extract described herein, for the manufacture of a pharmaceutical composition or medicament is also envisaged herein.

The term “pharmaceutical composition” particularly refers to a composition suitable for administering to a human. However, compositions suitable for administration to non-human animals are generally also encompassed by the term.

The pharmaceutical composition and its components (i.e. active agents and optional excipients) are preferably pharmaceutically acceptable, i.e. capable of eliciting the desired therapeutic effect without causing any undesirable local or systemic effects in the recipient. Pharmaceutically acceptable compositions of the invention may for instance be sterile or non-sterile. Specifically, the term “pharmaceutically acceptable” may mean approved by a regulatory agency or other generally recognized pharmacopoeia for use in animals, and more particularly in humans.

The barn dust extract is preferably present in the pharmaceutical composition in a therapeutically effective amount. By “therapeutically effective amount” is meant an amount of the active agent that elicits the desired therapeutic or prophylactic effect. Therapeutic efficacy can be determined by standard procedures, e.g. in test animals, e.g., ED₅₀ (the dose therapeutically effective in 50% of the population).

The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers and tonicity modifiers. It is within the knowledge of the skilled person to select suitable excipients for preparing the desired pharmaceutical composition of the invention. Exemplary carriers for use in the pharmaceutical composition of the invention include saline, buffered saline, dextrose, and water. Typically, choice of suitable excipients will inter alia depend on the specific active agent used, the disease to be treated, and the desired formulation of the pharmaceutical composition.

The exact dosage of the barn dust extract described herein (or the pharmaceutical composition comprising the same), will be ascertainable by one skilled in the art using known techniques. Suitable dosages provide sufficient amounts of the barn dust extract and are preferably therapeutically effective.

The compositions of the invention can be formulated in various forms, e.g. in solid, liquid, gaseous or lyophilized form and may be, for instance, in the form of a solution, an aerosol, a suspension, a lyophilisate, a powder, a tablet, a dragee, a suppository, a pill, a capsule, granule, an ointment, a cream, transdermal patches, a gel, suspensions, emulsions, syrups, liquids, elixirs, extracts, tincture or fluid extracts or in a form which is particularly suitable for the desired method of administration.

A variety of routes are applicable for administration of the composition according to the present invention. Typically, the composition may be prepared for nasal, inhalative, oral, conjunctival, subcutaneous, intraarticular, intraperitoneal, rectal, or vaginal administration. The composition may be in a form of a food additive, a food ingredient, or a composition suitable to be distributed in indoor air.

A barn dust extract of the invention or a composition of the invention, in particular a pharmaceutical composition, may be for use in the prevention or treatment of a disease. The use preferably comprises administering the barn dust extract or the composition to a subject that is preferably in need thereof. A barn dust extract of the invention or a composition of the invention, in particular a pharmaceutical composition, may be used for the manufacture of a composition for the prevention or treatment of a disease. The present invention also contemplates a method of preventing or treating a disease comprising administering a therapeutically effective amount of a barn dust extract of the invention or a composition of the invention to a subject.

The term “treatment” in all its grammatical forms includes therapeutic or prophylactic treatment of a subject in need thereof. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of diseases.

The disease may be selected from the group consisting of an allergic disease, a chronic inflammatory disease, and an autoimmune disease. Preferably, the disease is selected from the group consisting of hay fever, food allergy, asthma, urticaria, neurodermitis, atopy, including atopic sensitisation and atopic dermatitis, contact eczema, psoriasis, diabetes type 1 or 2, multiple sclerosis, rheumatoid arthritis, diseases of the thyroid gland, including Hashimoto Thyreoditis and Graves disease, preferably selected from the group consisting of atopy, including atopic sensitisation and atopic dermatitis, asthma and hay fever.

It is envisioned by the invention that the “subject” may be an animal, preferably a vertebrate, preferably a mammal. Preferred subjects include human, mouse, rat, rabbit, hamster, pig, dog, cat, cattle, sheep, goat, camel, monkey, or ape. It is envisioned by the invention that a human subject is preferred over other species, wherein a baby, and infant, or a pregnant woman is most preferred.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

### Example 1

### Method of Extract Preparation from Stable Dust

Barn dust extracts may be prepared, fractionized, lyophilized and analyzed according to following method.

Extract Preparation

1. Crushing barn dust using a mixer and with addition of dry ice.

2. Sieving of barn dust with two sieves (100 μm and 40 μm particle retention) (optional).

3. Suspending 3 g barn dust in 30 mL Millipore water (100 mg barn dust/liter)

4. Shaking suspension at 1000 rpm for two hours at room temperature.

5. Centrifugation of Suspension for 5 minutes at 2500×g at room temperature.

6. Filtration of supernatant with vacuum pump and cellulose filter (particle retention 5-13 μm). Use of multiple filters.

7. Second filtration with vacuum pump and cellulose filter (particle retention 2μm). Use of multiple filters.

8. Filtration with further filtration system (particle retention: 0.22 μm). Fractionation

9. Ultracentrifugation with 100 kDa membrane at 3000×g at room temperature (optional, only for preparation of 100/10 kDa fractions).

10. Ultracentrifugation with 10 kDa membrane at 3000×g at room temperature. Autoclaving

11. Autoclaving (20 min at 121° C., saturated steam). Lyophilization

12. Sterile filtration using syringe filters.

13. Dispensing 3 mL sample per vial under sterile conditions.

14. Lyophilization of samples Analysis

15. SEC analysis
a. dissolving sample in Millipore water.
b. measuring the sample (eluent: 0.15M NaCl, T 30° C., flow: 0.3 mL/min, combination of 130 Å und 300 Å column, wavelength: 210 nm)

### Example 2

### Effects of Lyophilization and/or Autoclaving on the Chromatographic Profile of a Total Stable Dust Extract and a Stable Dust Extract Comprising Fractions >10 kDa.

Unless otherwise stated, barn dust extracts were prepared as described in Example 1.

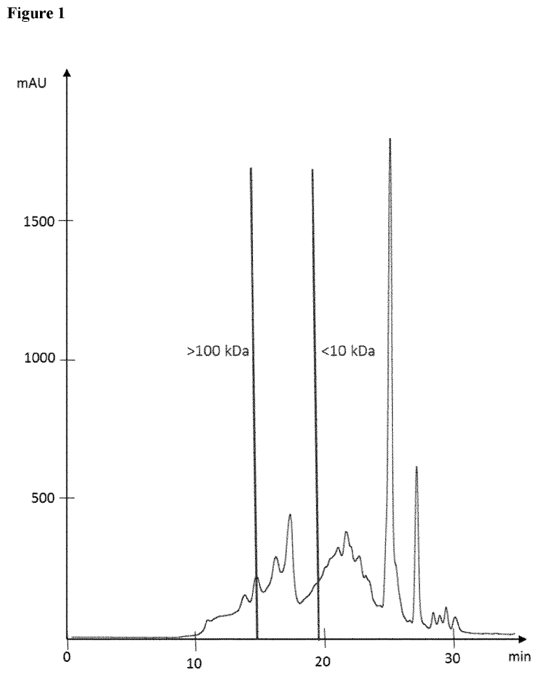

FIG. 1 shows a HPLC chromatogram of the stable dust total extract that has been not lyophilized and not autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter 4.6 mm, particle size 2.7 μm).

Column oven temperature: 30° C.

Mobile phase: 0.15 M NaCl with bidest. H₂O.

Flow rate 0.3 mL/min.

Injection volume: 10 μL.

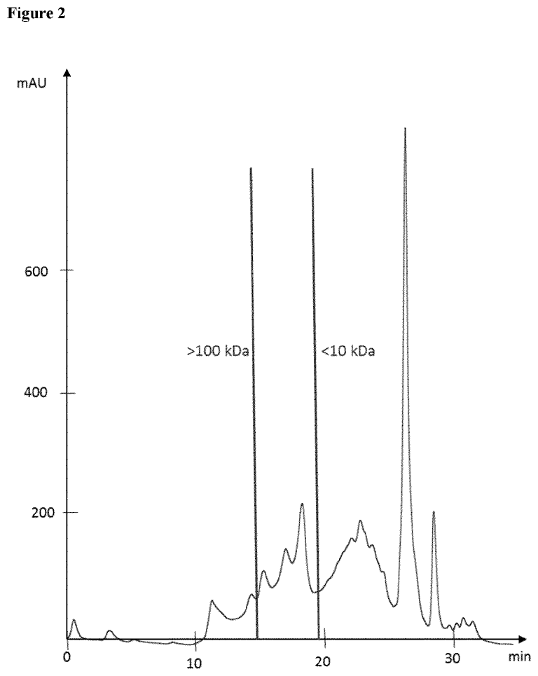

FIG. 2 shows a HPLC chromatogram of the stable dust total extract that has been lyophilized and not autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter 4.6 mm, particle size 2.7 μm).

Column oven temperature: 30° C.

Mobile phase: 0.15 M NaCl with bidest. H₂O.

Flow rate 0.3 mL/min.

Injection volume: 10 μL (12.3 mg/mL lyophilized dust in water).

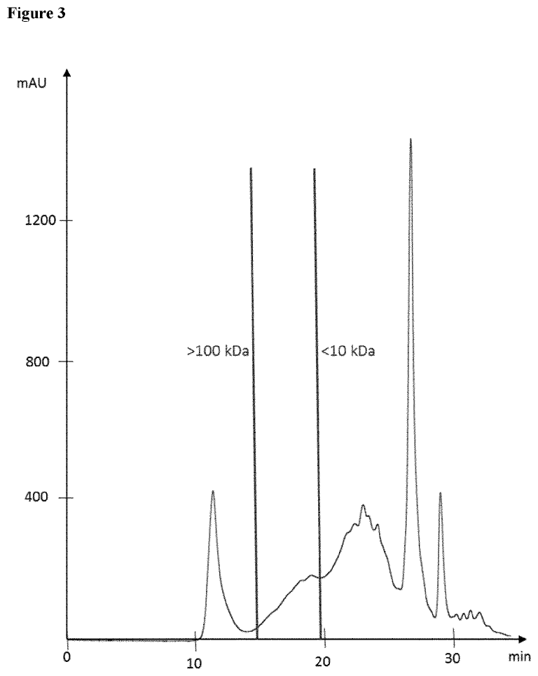

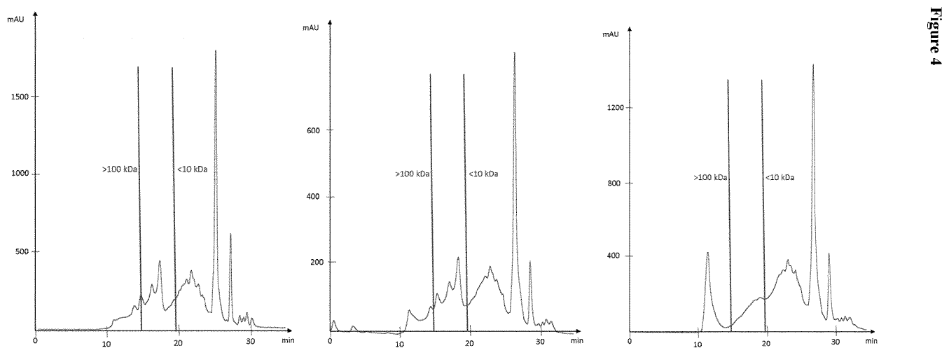

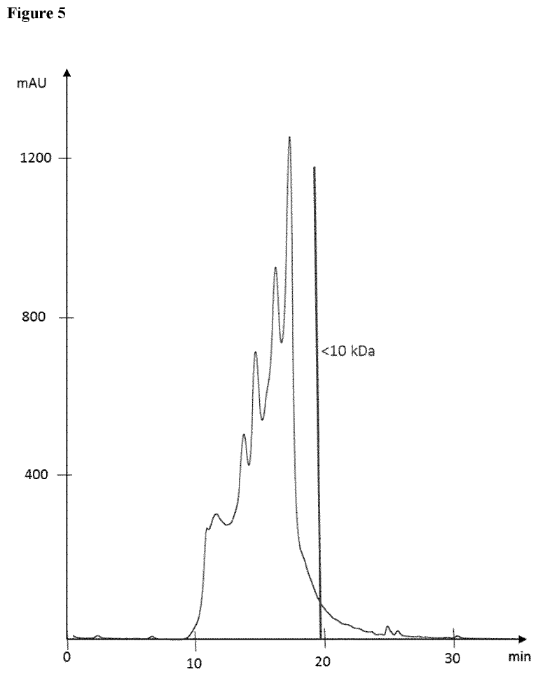

FIG. 3 shows a chromatogram of the stable dust Total extract that has been lyophilized and autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter 4.6 mm, particle size 2.7 μm)

Column oven temperature: 30° C.

Mobile phase: 0.15 M NaCl with bidest. H₂O.

Flow rate 0.3 mL/min.

Injection volume: 10 μL (9.9 mg/mL lyophilized dust in water).

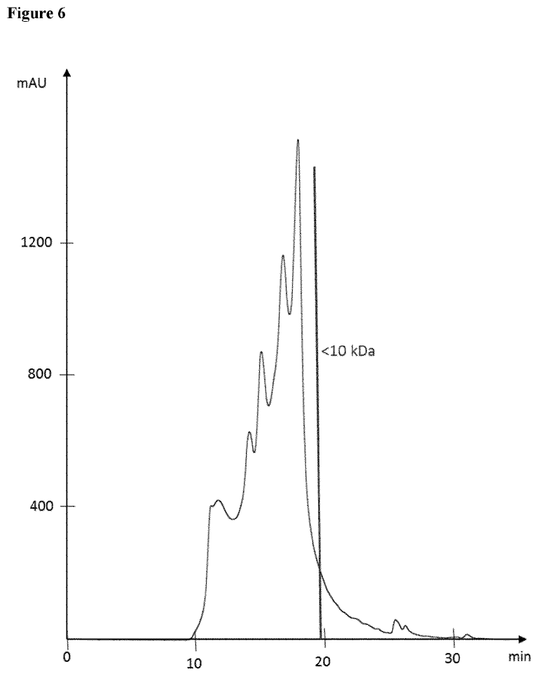

FIG. 5 shows a chromatogram of the >10 kDa fraction of a stable dust extract that has not been lyophilized and not autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter 4.6 mm, particle size 2.7 μm).

Column oven temperature: 30° C.

Mobile phase: 0.15 M NaCl with bidest. H₂O.

Flow rate 0.3 mL/min.

Injection volume: 10 μL.

FIG. 6 shows a chromatogram of the >10 kDa fraction of a stable dust extract that has been lyophilized and not autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter 4.6 mm, particle size 2.7 μm).

Column oven temperature: 30° C.

Mobile phase: 0.15 M NaCl with bidest. H₂O.

Flow rate 0.3 mL/min.

Injection volume: 10 μL (13.0 mg/mL lyophilized dust in water).

FIG. 7 shows a chromatogram of the >10 kDa fraction of a stable dust extract that has been lyophilized and autoclaved.

Equipment and parameters:

Stable dust: Hechfellner Hof 2017.

System: Agilent Series 1100.

Diode array detector: wavelength 210 nm.

Columns: Agilent Advance Bio SEC 130 Å+Agilent Advance Bio SEC 300 Å (each: length 30 cm, diameter 4.6 mm, particle size 2.7 μm).

Column oven temperature: 30° C.

Mobile phase: 0.15 M NaCl with bidest. H₂O.

Flow rate 0.3 mL/min.

Injection volume: 10 μL (8.4 mg/mL lyophilized dust in water).

### Example 3

### Change in Chromatographic Profile of a Non-Autoclaved and an Autoclaved Liquid Extract Over Time Under Different Storage Conditions (Room Temperature, 4° C. and −20° C.).

Unless otherwise stated, barn dust extracts were prepared as described in Example 1. Ultracentrifugation step 9 was omitted.

FIG. 9 illustrates the change in liquid extract (not autoclaved) measured after 8 days of storage under different conditions, namely storage at room temperature, refrigerated (storage at 4° C.) and freezed (storage at −20° C.).

FIG. 10 illustrates the change in peak height for the peaks of respectively 27 kDA, 5 kDA, 1 kDa, and 0.6 kDa, measured after 8 days of storage under different conditions, namely storage at room temperature, storage at 4° C. and storage at −20° C.

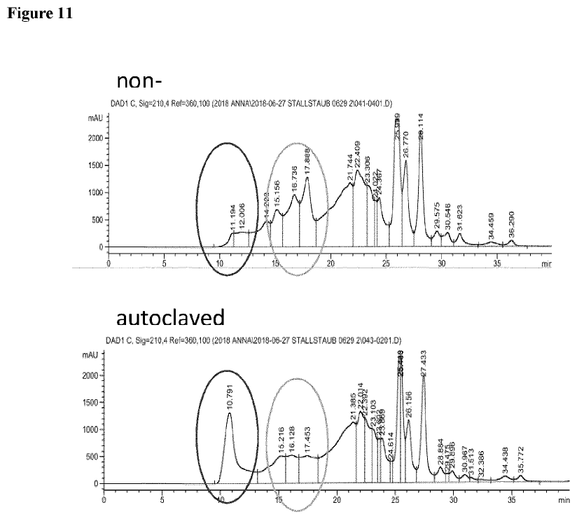

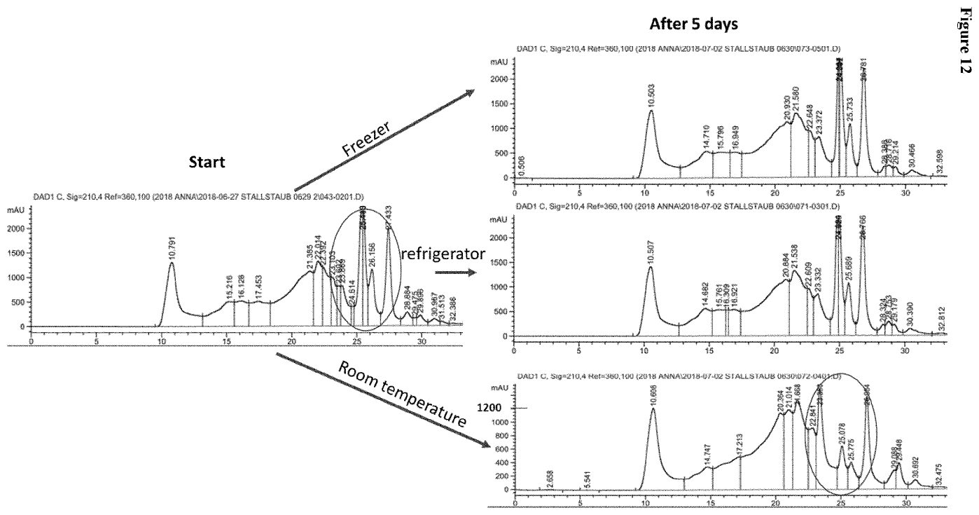

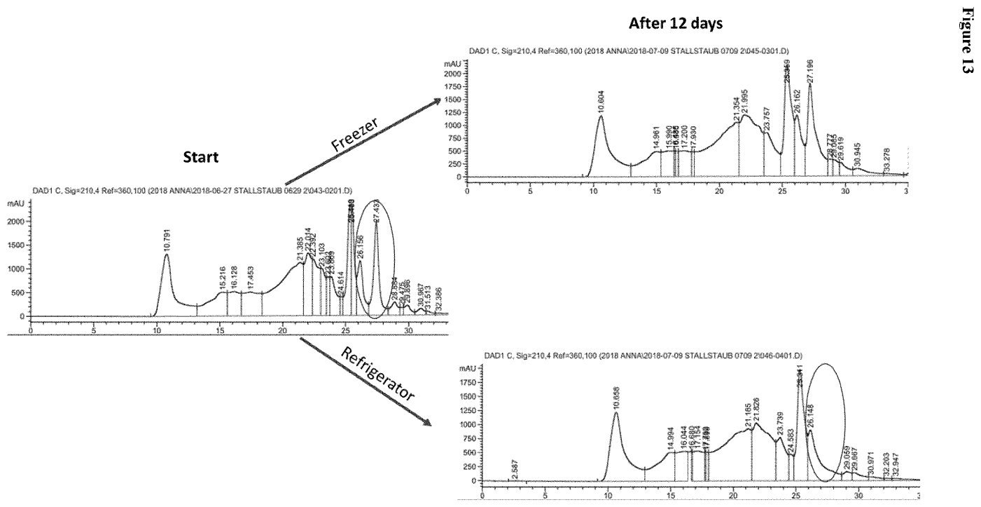

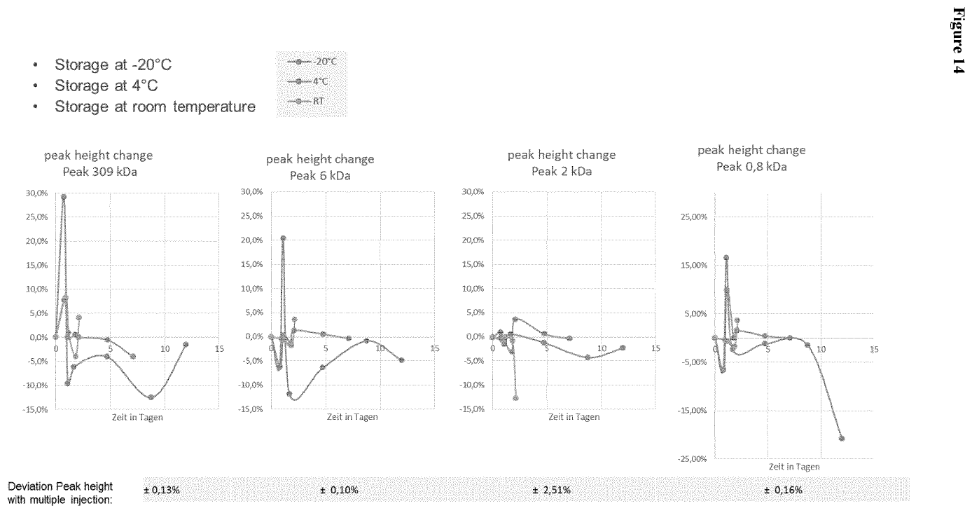

FIG. 11 illustrates the difference between non-autoclaved and autoclaved liquid extracts obtained from barn dust from Hechfellner Hof.

FIG. 12 illustrates the change in liquid extract (autoclaved) measured after 5 days of storage under different conditions, namely storage at room temperature, storage at 4° C. and storage at −20° C.

FIG. 13 illustrates the change in liquid extract (autoclaved) measured after 12 days of storage under different conditions, namely storage at room temperature, storage at 4° C. and storage at −20° C.

FIG. 14 illustrates the change in peak height for the peaks of respectively 309 kDa, 6 kDa, 2 kDa, and 0.8 kDa, measured after 8 days of storage under different conditions, namely storage at room temperature, storage at 4° and storage at −20°.

### Example 4

### Measurement of Eosinophil Production in Mice After Intra-Nasal Administration of Fractionated or Unfractionated Stable Dust Extracts

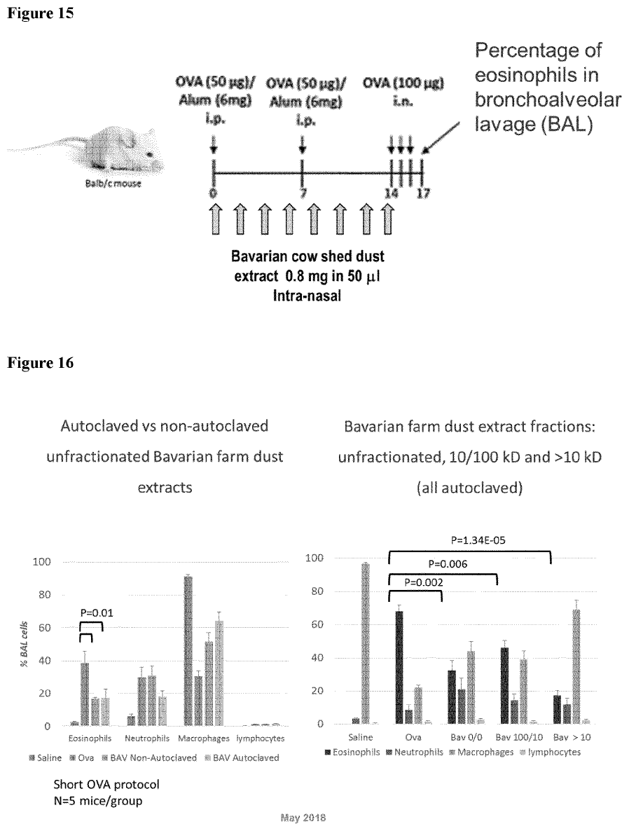

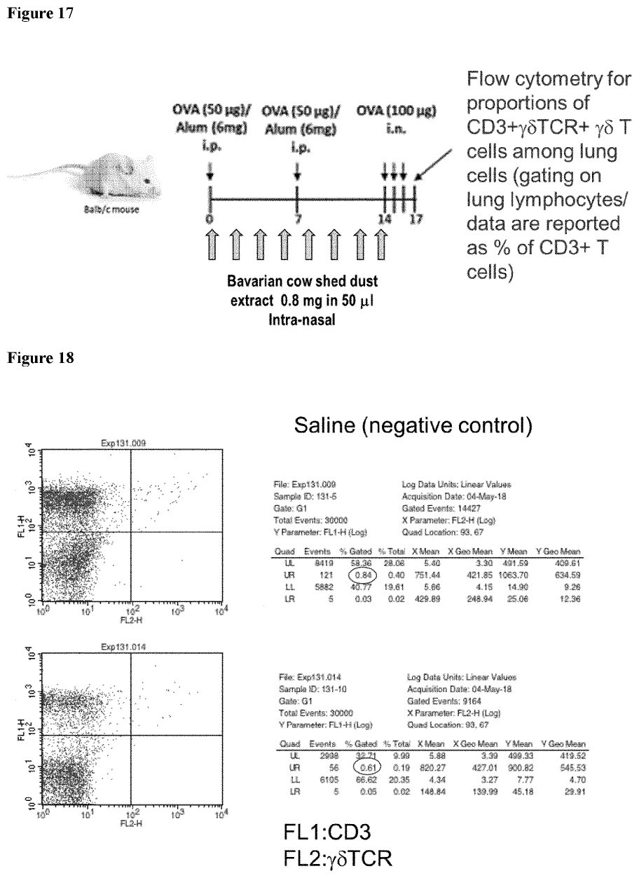

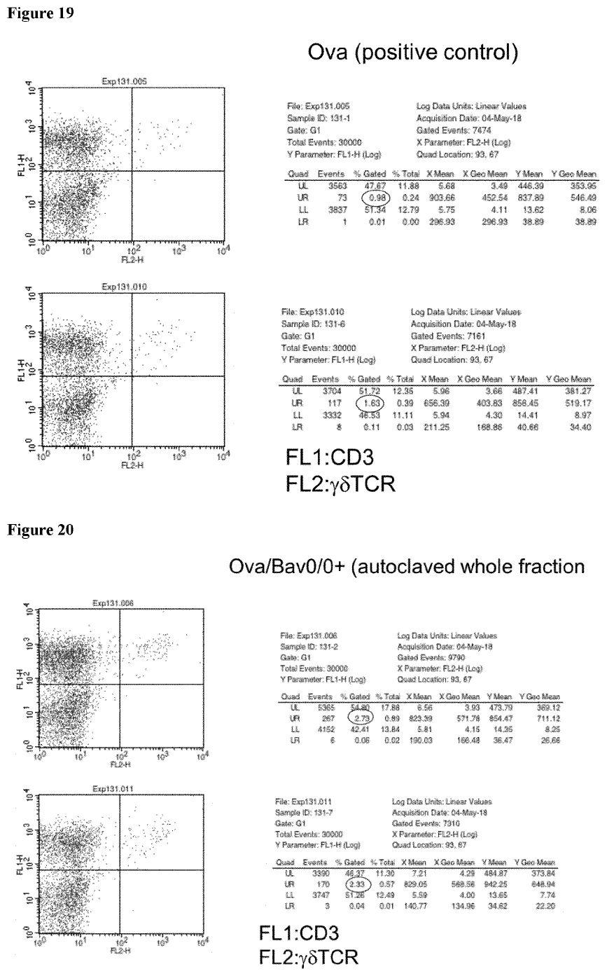

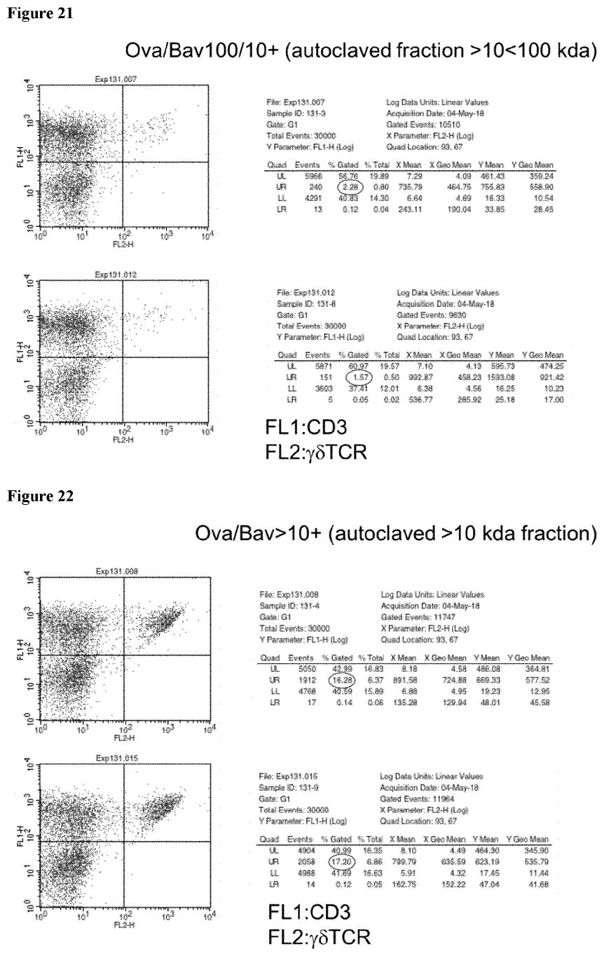

FIG. 15 illustrates the experimental setup used to measure eosinophil recruitment to the lung in mice after intra-nasal administration of fractionated or unfractionated stable dust extracts.

Dust extracts (0.8 mg dry weight in 50 μL/treatment) are instilled intra-nasally every 2-3 days for a total of 8 times beginning at day 0 into 7-week old Balb/c mice that are sensitized intra-peritoneally with 50 μg of OVA-Alum (6 mg) at day 0 and 7, and challenged intra-nasally with 100 μg OVA at day 14, 15, and 16. Broncho-alveolar lavage (BAL) is performed at day 17 by delivering cold 1% BSA in PBS (2 mL) into the airway via a tracheal cannula and gently aspirating the fluid. Cells are counted using a Countess II FL automated cell counter (Thermo Fisher Scientific) and differentials are determined by an operator blinded to mouse ID/grouping after staining with Hema 3 (Thermo Fischer Scientific) (at least 400 cells/slide). Statistical significance for BAL cellularity measurements is determined by an unpaired two tailed Student's t-test. P-values <0.05 are considered significant. Each treatment group includes a minimum of 5 mice

FIG. 16 (left panel) illustrates eosinophils count after treating mice with a saline solution (negative control), an allergen (OVA, positive control), unfractionated, autoclaved stable dust extract, and unfractionated, non-autoclaved stable dust extract.

FIG. 16 (right panel) illustrates eosinophils counts after treating mice with a saline solution (negative control), an allergen (OVA, positive control), unfractionated, autoclaved barn dust extract, fractions between 10 kDa and 100 kDa of autoclaved stable dust extract, and fractions bigger than 10 kDa of autoclaved stable dust extract.

### Example 5

### Measurement of γδ T Cell Production in Mice After Intra-Nasal Administration of Fractionated or Unfractionated Stable Dust Extracts

Mice are euthanized by lethal dose of anesthetic. Lungs are perfused and removed. To prepare single-cell suspension the lungs are minced with scissors and digested with 0.26 Wunsch U/mL of Liberase TM (Roche) and 4 U/ml of DNAse I (Sigma-Aldrich) at 37° C. for 1 h with shaking (60-70 rpm). The resulting suspension is passed through 20-22G needle and through the 70 μm cell strainer. After washing with complete medium, lung cells are resuspended in PBS/0.1% NaN₃/1.0% BSA at 5×10⁶ cells/mL. Cells are treated with Fc-Block for 10 min on ice and stained with fluorescently labeled antibodies to cell surface antigens for 30 min on ice in the dark. Cells are then washed and resuspended in PBS/0.1% NaN₃/1.0% BSA for flow cytometry.

The following antibodies to cell surface antigens are used: PE or BV510 conjugated hamster anti-mouse γδ T-Cell Receptor (GL3), FITC-hamster anti-mouse CD3c (145-2C11), PE-Cy7 or PE-Cy5-rat anti-mouse CD4 (RM4-5), APC-R700-rat anti-mouse CD8α (53-6.7). All from BD Pharmingen. Flow cytometry is performed on a FACSCalibur or LSRII (BD Sciences) flow cytometers. Data is collected on 10000-20000 events. Data analysis is performed using CellQuest (BD) or FlowJo (FlowJo) software. Proportions of γδ T cells are reported as % of CD3+ T cells gating on lung lymphocytes.

FIG. 18 illustrates the results of flow cytometry analysis to determine the proportion of CD3⁺γδ⁺ cells found in the lungs after treating mice with a saline solution (negative control). CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse.

FIG. 19 illustrates the results of flow cytometry analysis to determine the proportion of CD3⁺γδ⁺ cells found in the lungs after treating mice with an allergen (OVA). CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse.

FIG. 20 illustrates the results of flow cytometry analysis to determine the proportion of CD3⁺γδ⁺ cells found in the lungs after treating mice with allergen (OVA)+unfractionated, autoclaved barn dust extract. CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse.

FIG. 21 illustrates the results of flow cytometry analysis to determine the proportion of CD3⁺γδ⁺ cells found in the lungs after treating mice with allergen (OVA)+fractions of autoclaved stable dust extract between 10 kDa and 100 kDa. CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse.

FIG. 22 illustrates the results of flow cytometry analysis to determine the proportion of CD3⁺γδ⁺ cells found in the lungs after treating mice with allergen (OVA)+fractions of autoclaved stable dust extract >10 kDa. CD3⁺γδ T cells are positive for both CD3 staining (FL1, vertical axis) and γδ staining (FL2, horizontal axis) and are located in the upper right quadrants of the two panels on the left. Each panel presents data from a different mouse (n=2). The two panels on the right present quantitative analyses for the corresponding panels on the left. Circled in red are the proportions of γδ T cells among CD3⁺ T cells found in each mouse.

The invention illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims.

1. A method of preparing a barn dust extract, comprising:
(d) providing a mixture comprising a barn dust and a liquid;
(e) isolating a fraction of the mixture, wherein the barn dust extract comprised in the fraction consists essentially of molecules having a molecular weight of at least about 5 kDa, preferably at least about 10 kDa.

2. The method of claim 1, wherein the barn dust is from a farm.

3. The method of claim 1 or 2, wherein the barn dust is from a cow barn.

4. The method of any one of the preceding claims, wherein the liquid is water, an aqueous solution, or a water-miscible solvent.

5. The method of claim 4, wherein the aqueous solution is a solution of sodium chloride, preferably normal saline.

6. The method of any one of the preceding claims, wherein step (e) comprises enriching the fraction by factor of at least about 5, preferably at least about 10, preferably at least about 20, preferably at least about 100.

7. The method of any one of the preceding claims, wherein step (e) comprises depleting a fraction wherein the barn dust extract comprised therein consists essentially of molecules having a molecular weight of at most about 5 kDa, preferably at most about 10 kDa by factor of at least about 5, preferably at least about 10, preferably at least about 20, preferably at least about 100.

8. The method of any one of the preceding claims, further comprising the step of (f) subjecting the mixture to heat treatment at a temperature of at least about 110° C. and/or a pressure of at least about 1.5 bar for at least about 3 min.

9. The method of claim 8, wherein step (f) comprises subjecting the mixture to a saturated steam.

10. The method of any one of the preceding claims, further comprising:
(g) drying the fraction obtained in step (e).

11. The method of claim 10, wherein step (g) is conducted by lyophilization or spray drying.

12. The method of any one of the preceding claims further comprising:
(a) collecting barn dust.

13. The method of any one of the preceding claims further comprising
(b) mixing barn dust with a liquid.

14. The method of any one of the preceding claims further comprising
(c) depleting particles having a size of at least about 0.2 μm.

15. The method of claim 14, wherein step (c) comprises one or more filtration steps.

16. The method of claim 14 or 15, wherein step (c) comprises depleting the particles having a size of at least about 0.2 μm by factor of at least about 10, preferably at least about 100, preferably at least about 1000.

17. The method of any one of the preceding claims, wherein the barn dust extract that consists essentially of molecules having a molecular weight of at least about 5 kDa comprises molecules having a molecular weight of at least about 100 kDa.

18. The method of any one of the preceding claims, wherein the barn dust extract essentially consists of water-soluble molecules.

19. A barn dust extract obtainable by the method of any one of claims 1 to 18.

20. A barn dust extract consisting to at least about 90% by dry matter of molecules having a molecular weight of at least about 5 kDa, preferably at least about 10 kDa, wherein a size exclusion chromatogram of the barn dust extract has in the fraction having a molecular weight of at least about 10 kDa two characteristic peaks A and B, wherein
(a) peak A has its maximum in the range of 35 to 75 kDa; and
(b) peak B has its maximum in the range of 300 to 700 kDa.

21. The barn dust extract of claim 20, wherein size exclusion chromatography is conducted using an Agilent Advance Bio SEC 130 A and/or 300 A column and a 0.15 M NaCl solution as mobile phase, and wherein particles are detected by an optical detector at a wavelength of 210 nm.

22. The barn dust extract of claim 20 or 21, wherein the barn dust extract has undergone heat treatment as defined in claim 8 or 9.

23. The barn dust extract of any one of claims 19 to 22, wherein the barn dust extract consists to at least about 75%, preferably 80%, preferably 85%, preferably 90%, preferably 95%, preferably 98%, preferably 99%, preferably 99.5%, preferably 99.9% by dry matter of molecules having a molecular weight of at least about 5 kDa.

24. The barn dust extract of any one of claims 19 to 23, wherein the barn dust extract consists to at least about 75%, preferably 80%, preferably 85%, preferably 90%, preferably 95%, preferably 98%, preferably 99%, preferably 99.5%, preferably 99.9% by dry matter of molecules having a molecular weight of at least about 10 kDa.

25. A composition comprising the barn dust extract of any one of claims 19 to 24.

26. The composition of claim 25, wherein the composition is a pharmaceutical composition.

27. The composition of claim 25 or 26, wherein the composition is in the form of a solution, an aerosol, a suspension, a lyophilisate, a powder, a tablet, a dragee, or a suppository.

28. The composition of any one of claims 25 to 27, wherein the composition is for nasal, inhalative, oral, conjunctival, subcutaneous, intraarticular, intraperitoneal, rectal, or vaginal administration.

29. The composition according to any one of claims 25 to 28, wherein the composition is a food additive, a food ingredient, or a composition suitable to be distributed in indoor air.

30. The barn dust extract of any one of claims 19 to 24 or the composition of any one of claims 25 to 29 for use in the prevention or treatment of a disease.

31. The barn dust extract or the composition for the use of claim 30, wherein the disease is selected from the group consisting of an allergic disease, a chronic inflammatory disease, and an autoimmune disease.

32. The barn dust extract or the composition for the use of claim 31, wherein the disease is selected from the group consisting of hay fever, food allergy, asthma, urticaria, neurodermitis, atopy, including atopic sensitisation and atopic dermatitis, contact eczema, psoriasis, diabetes type 1 or 2, multiple sclerosis, rheumatoid arthritis, diseases of the thyroid gland, including Hashimoto thyreoditis and Graves disease, preferably selected from the group consisting of atopy, including atopic sensitisation and atopic dermatitis, asthma and hay fever.

33. The barn dust extract or the composition for the use of any one of claims 30 to 32, wherein the use comprises administering the barn dust extract or the composition to a subject.

34. The barn dust extract or the composition for the use of claim 33, wherein the subject is human.

35. The barn dust extract or the composition for the use of claim 34, wherein the subject is a baby, and infant, or a pregnant woman.

36. Use of the barn dust extract of any one of claims 19 to 24 for the manufacture of a composition for the prevention or treatment of a disease.

37. A method of preventing or treating a disease comprising administering a therapeutically effective amount of a barn dust extract of claims 19 to 24 to a subject.
